(19) United States
(12) Patent Application Publication
(10) Pub. No.: US 2014/0180029 A1
(43) Pub. Date: Jun. 26, 2014

(54) ELECTRODE ARRAY FOR ELECTROMYOGRAPHIC MEASUREMENTS

(71) Applicants: Hans-Ullrich Hansmann, Barnitz (DE); Marcus Eger, Lubeck (DE); Thomas Krüger, Reinfeld (DE); Lorenz Kahl, Lubeck (DE)

(72) Inventors: Hans-Ullrich Hansmann, Barnitz (DE); Marcus Eger, Lubeck (DE); Thomas Krüger, Reinfeld (DE); Lorenz Kahl, Lubeck (DE)

(73) Assignee: DRAGER MEDICAL GMBH, Lubeck (DE)

(21) Appl. No.: 14/117,502

(22) PCT Filed: Nov. 14, 2011
(86) PCT No.: PCT/EP2011/005732

§ 371 Date: Nov. 13, 2013

(30) Foreign Application Priority Data
May 13, 2011 (DE) 10 2011 101 580.2

(51) Int. Cl. A61B 5/0492; A61B 5/113

(52) U.S. Cl. CPC A61B 5/0492; A61B 5/113
USPC 600/301; 600/393

(57) ABSTRACT

A sensor device for the electromyographic recording of muscle signals on the skin of a living body includes at least two recording electrodes and an earth electrode. The electrodes have a common carrier layer that has at least one perforation at which the carrier layer can be separated. After the separation of the carrier layer at the perforation, each electrode is located separately on a separated part of the carrier layer. Further, the sensor device includes at least one shielded cable, one end of which is connected to one of the electrodes and the other end of which is connected to a contact element. The contact element can be connected to an evaluation unit by means of a connecting element such that signals can be transmitted to the evaluation unit.

### CROSS REFERENCE TO RELATED APPLICATIONS

This application is a United States National Phase Application of International Application PCT/EP2011/005732 filed Nov. 14, 2011 and claims the benefit of priority under 35 U.S.C. §119 of German Patent Application DE 10 2011 101 580.2 filed May 13, 2011, the entire contents of which are incorporated herein by reference.

### FIELD OF THE INVENTION

The present invention pertains to a sensor device for electromyographic detection of muscle signals on the skin of a live body, which [device] comprises at least two lead electrodes as well as a ground electrode. Furthermore, the present invention pertains to a method as well as a system in which such a sensor device is used.

### BACKGROUND OF THE INVENTION

In modern medicine, especially, the method of electromyography is used for the electrodiagnostic procedures of muscle diseases and muscle disorders. Signals detectable by measuring technology, which are fluctuations of potential in the muscle which reflect muscle activity, are recorded in this method. During an electromyographic examination, the electrical activity in the resting and in the contracted muscle is measured and then compared with normal values. For this, the resting muscle is stimulated after the measurement in order to achieve a contraction of the muscle, whose resulting electrical activity is also recorded. The method of electromyography includes the detection, analysis and recording of muscle potentials in an electromyogram (EMG).

The detection of muscle potentials is carried out via one or more electrodes. For example, needle electrodes, which are inserted into the muscle to be examined, are used. Needle electrodes can be placed in a point-focal manner and especially detect the muscle potential of individual muscle fibers. Surface electrodes, with which especially muscle potentials of muscle groups are detectable, are a different form of electrodes. For the detection of the muscle potentials, the surface electrodes are simply adhered to the skin of the patient over the muscle to be examined. An EMG recorded by means of surface electrodes is known in medicine also as SEMG (surface electromyogram).

SEMG signals have their origin in the electrical activity of muscle fibers, which electrical activity is determined by neuronal processes, which fibers are a component of a motor unit as the smallest functional unit for describing the neuronal control of muscle contraction. Another component of a motor unit is a motoneuron. If a motoneuron is active, neurotransmitters, for example, acetylcholine, which lead to a local depolarization of the muscle fiber, are released in the synaptic gap between the neuronal end-plate of the motoneuron and the associated muscle fiber. The action potential developed in this manner reproduces along the muscle fiber in both directions and results in contraction of the muscle fiber. The space-time activation of many muscle fibers can then be detected as an electrical signal on the skin by means of the surface electrodes.

For electromyography, besides the diagnosis of muscle diseases and muscle disorders, there are a large number of other possible applications such as, for example, determination of nerve conduction velocities in neurology (also known as electroneurography), determination of biofeedback in muscle training or optimization of movements of athletes in physical therapy or biomechanics as well as the control of prostheses.

However, electromyography is also increasingly used for the control of respirators. An EMG of the most important respiratory muscle with inspiratory action, the diaphragm, which was recorded either by means of needle electrodes or esophageal probe or else by means of surface electrodes, is used as the basis for the control. Needle electrodes have, however, the drawback that the insertion, as an invasive procedure in a human body, is rated as having the corresponding, associated risks, for which specific medical knowledge is required. The same is true for placing an esophageal probe as well. Moreover, both the insertion of needle electrodes and the placement of an esophageal probe represent a corresponding additional stress for the patient.

On the other hand, a use of surface electrodes has especially the advantage that risks and additional stress factors for a patient can be prevented due to their noninvasive use. Moreover, the use of surface electrodes is generally known in medicine, since such surface electrodes are used, for example, for recording an electrocardiogram (ECG), in which the sum of electrical activities of all heart muscle fibers is recorded. ECG recordings belong to medical practice and are performed very frequently.

For recording an SEMG, usually individual surface electrodes are positioned as lead electrodes on the body of a patient and then adhered to the skin of the patient. Here, one of the surface electrodes is usually used as a ground electrode for grounding the patient as well as for reducing artefacts in the SEMG and for creating electrically defined lead conditions. The surface electrodes are then each connected to an analysis unit via individual electrical cables. Both the handing of individual surface electrodes and assignment of the surface electrodes to the respective electrical cables and thus to the individual lead positions after the positioning of the surface electrodes often proves to be difficult, however, without instructions.

Sensor devices, which simplify the handling of surface electrodes as well as their assignment to the associated lead position, are therefore known from the state of the art. Thus, for example, document DE 692 30 191 T2 describes a multiple electrode strip, which is configured for a certain positioning of a plurality of surface electrodes for the detection of bioelectric signals. The strip has a plurality of surface electrodes, which are connected to one another via a folded, multi-channel conduction strip, so that an adaptation to the physique of a patient is possible.

However, a drawback of this strip is that the surface electrodes are arranged one behind the other on the strip. The surface electrodes can thus always be positioned only on a line predetermined by the folded conduction on the body of a patient. Deviations from this line are thus only possible to a limited extent, so that a positioning is difficult and laborious to some extent. This effect is increased all the more, the wider the folded conduction strip has to be stretched because of the physique of the patient. Moreover, the folded conduction strip is broader than a conventional cable, which may lead to skin irritations when placing the folded conduction strip on the body of the patient.

### SUMMARY OF THE INVENTION

Hence, one object of the present invention is to provide a sensor device for the electromyographic detection of muscle signals, which device is simple to handle, with which the surface electrodes can be positioned in a variable manner on the body and which compromise the patient only a little. Another object of the present invention is to provide a method and a system, in which such a sensor device is used.

The present invention provides a sensor device for electromyographic detection of muscle signals on the skin of a live body, which device comprises at least two lead electrodes as well as a ground electrode. The electrodes have a common support layer, which comprises at least one perforation, at which the support layer can be separated. After separating the support layer at the perforation, each electrode is located separately on a separated section of the support layer. Furthermore, the sensor device has at least one shielded cable, one end of which is connected to one of the electrodes and the other end of which is connected to a contact element. The contact element can be connected to an analysis unit by means of a connecting element, such that signals are transferable to the analysis unit.

A live body in the sense of the present invention may be both a human body and an animal body.

Due to the arrangement of the electrodes on a common support layer, all electrodes needed for the recording of an SEMG are simultaneously available to the user of the sensor device. The support layer can then be separated at the perforation, so that individual electrodes separated one after the other arise due to the separation, which can be positioned on the body of a patient as needed without position limitations. Moreover, the electrodes are already connected to a contact element via at least one cable, so that only another connection of the contact unit to the analysis unit can be established. Thus, the fastening of individual cables to the electrodes is also omitted. Moreover, the cable has only a small diameter, so that the patient is not or only slightly compromised when placing the cable after placing the sensor device on his body.

In one embodiment, the sensor device has at least one mechanical sensor, which is designed such that it can pick up at least one geometric change in the live body.

A geometric change in a human body is caused, for example, during the breathing of the patient by the raising and lowering of his thorax. Thus, the sensor picks up mechanical changes in the skin of the patient, which are cyclically triggered due to the change in the geometry of the thorax and of the abdomen during breathing. Since both the SEMG signals and the signals of the mechanical sensor lie in the range of a few mV and thus are very small, they can be overlapped and easily affected by other signals, for example, due to external electromagnetic fields and hence are subject to artefacts. An influence of both signals by internal interference signals, for example, due to the heart muscle signal in the recording of an SEMG on the thorax, is also possible. Interference signals, which are also known as ‘crosstalk,’ may, however, lead to artefacts in the SEMG, which make an analysis difficult or possibly even impossible. Combining the sensor device with a mechanical sensor now has the advantage that such artefacts can be better recognized and suppressed. Furthermore, this combination provides additional information about the status of the respiratory muscles, such as, for example, the degree of fatigue or efficiency. Moreover, a reliable recognition of the two breathing phases, inspiration and expiration, is possible with this combination.

A variant of the sensor device is characterized in that the mechanical sensor is arranged between the two lead electrodes.

The arrangement of the sensor between the two lead electrodes has the advantage that respiration belts, which are usually used in medical practice, which record and measure a change in length and thus recognize a geometric change in the live body due to respiration, are dispensed with. This is associated both with a greater degree of movement for the patient during the entire monitoring time and with a prevention of skin irritations due to the belt lying on the skin of the patient. Moreover, the laborious placement of the respiration belt, which is usually associated with a position movement of the patient, is dispensed with for the care staff.

In one embodiment of the sensor device, the mechanical sensor is designed as a strain sensor. In another embodiment of the sensor device, the mechanical sensor is designed as a piezoelectrode element.

Both embodiments are especially suitable for the measurement of very small geometric changes in a live body caused by breathing, so that it is possible to recognize early when difficulties in breathing arise, in which the thorax and abdomen are no longer completely raising and/or lowering.

In one embodiment of the sensor device, each electrode is individually connected by means of a shielded cable to the contact element. In another embodiment of the sensor device, the cable is guided from one electrode to the next electrode. Here, the cable is connected to each electrode and designed as a multiwire cable.

In both embodiments, an assignment of the individual electrodes to their respective lead positions is no longer necessary, since the cable is already integrated into the sensor device. Thus, the mix-up of cables during the mounting at the individual electrodes is effectively prevented. Moreover, the mechanical stress of the electrode-patient adhesive surface after adhering of the electrodes, which usually occurs during a subsequent connection of the individual electrodes to separate cables, is also eliminated. Furthermore, a knotting or intertwining of the individual cables is effectively prevented.

A variant of the sensor device is characterized in that the cable is laid, such that after separation of the support layer at the perforation, an additional length of the cable can be released.

The release of an additional length of the cable makes possible a variable adaptation of the sensor device to the different physique of patients. Thus, a use of the sensor device regardless of body size and physique of the patient is possible. The sensor device is thus suitable both for children and for large and small adults.

In one embodiment of the sensor device, the cable is laid in a meander-shaped pattern.

With this embodiment a longer cable length can be made available for a greatest possible variability in the adaptation of the sensor device to the different physique of patients, without a compact construction of the sensor device being thereby compromised.

In one embodiment of the sensor device, at least one pictogram is arranged on a top side of the support layer. In a variant of the sensor device, the pictogram images a positioning of the electrodes on a live body and/or an assignment of the electrodes to a lead position.

Since SEMG recordings are not a standard examination method in medical practice, as are ECG recordings, for example, a pictogram facilitates and simplifies both the positioning of the electrodes on the body of a patient and the assignment of the electrodes to a lead position. With an accurate positioning of surface electrodes on the body of the patient directly over the muscle to be examined, the SEMG signals can be better detected, and interference signals, which appear as artefacts in an SEMG, can thus be at least partly suppressed. The correct assignment of the electrodes to a lead position is in turn a prerequisite for a correct evaluation and interpretation of the SEMG as a basis for making a diagnosis for the treatment of a patient.

One embodiment of the sensor device is characterized in that the contact element is arranged above an electrode, especially above the ground electrode.

An even more compact construction of the sensor device is achieved by means of this arrangement. Moreover, the patient is not additionally stressed with another adhesive surface. Here, the contact element is arranged on the side of the ground electrode, which is directed away from the patient and lies opposite the adhesive surface of the ground electrode.

In a variant of the sensor device, the electrodes are color coded.

A color coding of the electrodes makes possible a simple, certain and fast assignment of the electrodes to the individual lead positions and reliably prevents a mix-up of the positions.

The present invention also provides a method for the electromyographic detection of muscle signals on the skin of a live body with at least two lead electrodes as well as one ground electrode. In the method, all electrodes are arranged together on a support layer, and the support layer is separated at least one perforation, such that after the separation, each electrode is located separately on a separated section of the support layer. Furthermore, in the method, one end of a shielded cable is connected to one of the electrodes and the other end of the shielded cable is connected to a contact element and the contact element is connected to an analysis unit by means of a connecting element, such that signals can be transferred to the analysis unit.

In one embodiment of the method, an additional length of cable is released after separation of the support layer at the perforation.

Furthermore, the present invention provides a system for the electromyographic detection of muscle signals on the skin of a live body, which [system] comprises an above-described sensor device as well as an analysis unit, to which the sensor device is connected.

The present sensor device makes possible an accurate positioning of surface electrodes on the body of a patient for the recording of an SEMG. Moreover, the assignment of the surface electrodes to the respective lead positions proves to be very simple with this sensor device, so that mix-ups of the positions are effectively prevented. The sensor device can be used without secondary knowledge about the analysis unit, since no individual electrical connections have to be established. Thus, a knotting or intertwining of the individual cables among each other is also prevented.

The above-mentioned and other advantages, special features and advantageous variants of the present invention are also evident based on the exemplary embodiments, which are described below with reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of a surface electrode;

FIG. 2a is a sectional view A-A through a first sensor device;

FIG. 2b is a sectional view B-B through a second sensor device;

FIG. 3a is a top side view of a support layer of the first sensor device with cabling;

FIG. 3b is a top side view of a support layer of the second sensor device with cabling;

FIG. 4a is a view showing a pictogram on the top side of the support layer of the first sensor device;

FIG. 4b is a view showing a pictogram on the top side of the support layer of the second sensor device;

FIG. 5a is a top view showing a top side of the support layer of the first sensor device with cabling and pictogram;

FIG. 5b is a top view showing a top side of the support layer of the second sensor device with cabling and pictogram;

FIG. 6a is a side view showing a strain sensor as mechanical sensor between two electrodes; and

FIG. 6b is a side view showing a piezoelectric sensor as a mechanical sensor between two electrodes.

### DESCRIPTION OF THE PREFERRED EMBODIMENTS

Referring to the drawings in particular, for the recording of an SEMG, surface electrodes are used for measuring SEMG signals on the skin of a patient. Silver-silver chloride gel electrodes, which are known from the state of the art, are used here, for example. Such surface electrodes are favorable and available everywhere, since they are also used, for example, for ECG recordings.

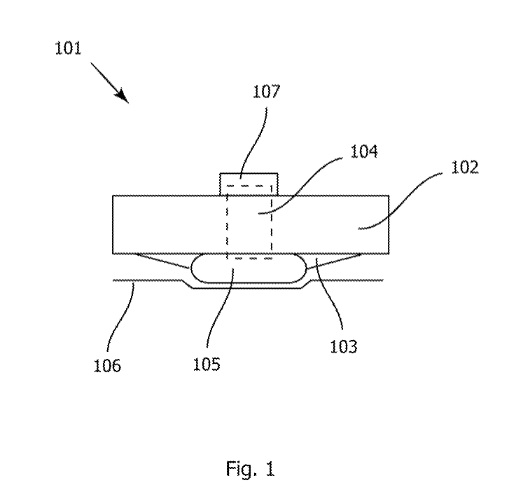

FIG. 1 schematically shows the example of a surface electrode 101, designated below also as just electrode 101. The electrode 101 has a support layer 102, on the underside of which an adhesive surface 103 is applied. With this adhesive surface 103, the electrode 101 can be adhered to the skin of a patient, so that the electrode 101 cannot slip during a signal recording.

The support layer 102 is made of, for example, nonwoven fabric, foam or foil. There are various design possibilities for the adhesive surface 103. When only a slight covering of the skin surface by the adhesive surface 103 is possible or preferred, smaller surfaces of approximately 2 cm² may be provided for the adhesive surface 103, for example. For this, highly adhesive adhesives have to be used, which may possibly lead to skin irritations. If, on the other hand, a larger covering of the skin surface is possible or preferred, then slightly adhesive adhesives offer a better tolerance. Moreover, motion artefacts between the electrode 101 and the skin of the patient can thus be better prevented.

The electrode 101 has, furthermore, a lead element 104, via which the signal detection is achieved. On the underside of the lead element 104 is located a conductive gel 105, which guarantees a good contact of the lead element 104 to the skin of the patient and which passes on the signal from the skin of the patient to the lead element 104. As an alternative, the adhesive surface 103 can be designed as a conductive adhesive surface, so that the conductive gel 105 is not necessary. For both forms of the electrode 101, it is essential that the conductive surface should have a size between approximately 0.1 cm² and 3 cm². The surface should preferably have a size below 1 cm².

During the mounting and transport of the electrode 101, the adhesive surface 103 and conductive gel 105 are protected by a protective layer 106, which is made of, for example, paper or foil, which can be simply pulled off before using the electrode 101.

A contact 107, via which an electrical connection to an analysis unit can be established, is arranged above the lead element 104. Shielded electrical cables, which can be connected to the contact 107, for example, by means of push-button, clip or clamp, are usually used for this. A fixed connection of the electrical connection to the contact 107 is likewise possible.

The above-described general structure of the electrode 101 is characteristic for the structure of all electrodes described below.

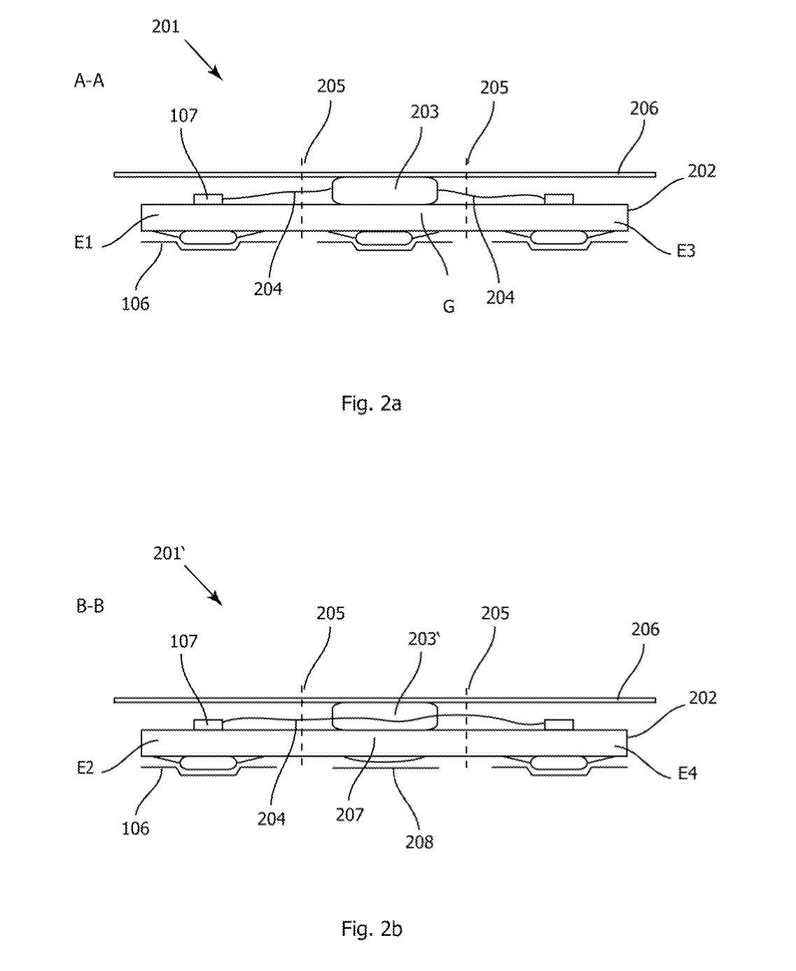

FIG. 2a schematically shows the example of a section A-A through a first sensor device 201. The sensor device 201 comprises a common support layer 202 for the electrodes E1 and E3, in which they are lead electrodes for recording an SEMG. The electrode G is designed as ground electrode likewise in the support layer 202. In the sensor device 201, the conductive electrode surfaces of the electrodes E1, E3 and G have each an area of approximately 1 cm² and the adhesive surfaces of the electrodes E1, E3 and G have each an area of approximately 8 cm². This applies generally also to all electrodes described below.

Above electrode G is arranged a contact element 203, which is adhered, for example, to electrode G and with which electrode G is connected directly and the electrodes E1 and E3 are each electrically connected via cables 204. Here, the cables are permanently connected both to the electrodes E1 and E3 and to the contact element 203. However, provisions may also be made for the cables 204 to be connected via detachable contacts to the electrodes E1 and E3 as well as to the contact element 203. One or more adhesive surfaces, which are not shown, with which the cables can be fixed on the skin of a patient, can each be attached to the cables 204. As cables 204, commercially available shielded electrical cables may be used, which are permitted in the field of medical technology.

All electrical connections to the electrodes E1, E3 and G are brought together in the contact element 203, such that only an electrical connection of the contact element 203 to an analysis unit still has to be established. This can take place, for example, via a shielded electrical cable or even via a plug-in connection. With the bringing together of all electrical connections in the contact element 203, all electrodes E1, E3 and G thus no longer have to be connected to the analysis unit individually. As a result, the use of the sensor device 101 is simplified and a mix-up of cables during the connection to the analysis unit is prevented.

The support layer 202 of the sensor device 201 has a perforation 205, at which the support layer 202 can be separated into a plurality of sections. The perforation 205 is formed in the support layer, such that after separating the support layer 202 at the perforation 205, each electrode E1, E3 and G is located on each section of the support layer 202. A separation of the electrodes E1, E3 and G, which makes possible a variable positioning of the electrodes E1, E3 and G on the body of a patient, is thus achieved with the perforation 205.

A covering 206, which especially correspondingly protects the cables 204 during the mounting and the transport of the sensor device 201 and which adheres adhesively to the contacts 107 and/or to the contact element 203, can be provided above the support layer 202. If desired, the covering 206 may have a perforation 205, which coincides with the perforation 205 of the support layer 205, so that the contacts 207 of the electrodes E1, E3 and G are further protected even after the separation. Possible materials for the covering 206 are, for example, paper or foil.

Even though all electrodes E1, E3 and G are shown in FIG. 2a protected separately with their own protective layer 106, it is possible to protect all electrodes E1, E3 and G with one protective layer, which jointly covers all electrodes E1, E3 and G. This protective layer may then likewise have a perforation 205, which coincides with the perforation 205 of the support layer 202, so that after the separation, the electrodes E1, E3 and G are also still individually provided with a protective layer until they shall be adhered to the skin of a patient.

FIG. 2b schematically shows the example of a section B-B through a second sensor device 201′. The sensor device 201′ comprises a support layer 202 with the electrodes E2 and E4 and has a contact element 203′, which is fastened on the top side of the support layer 202, as it is adhered, for example, to the support layer 202. The support layer 202 under the contact element 203′ is designed as an adhesive pad 207, with which the contact element 203′ can be adhered to the skin of a patient. The adhesive surface of the adhesive pad 207 is protected by a protective layer 208, which is comparable to the protective layer 106 of the electrodes E2 and E4.

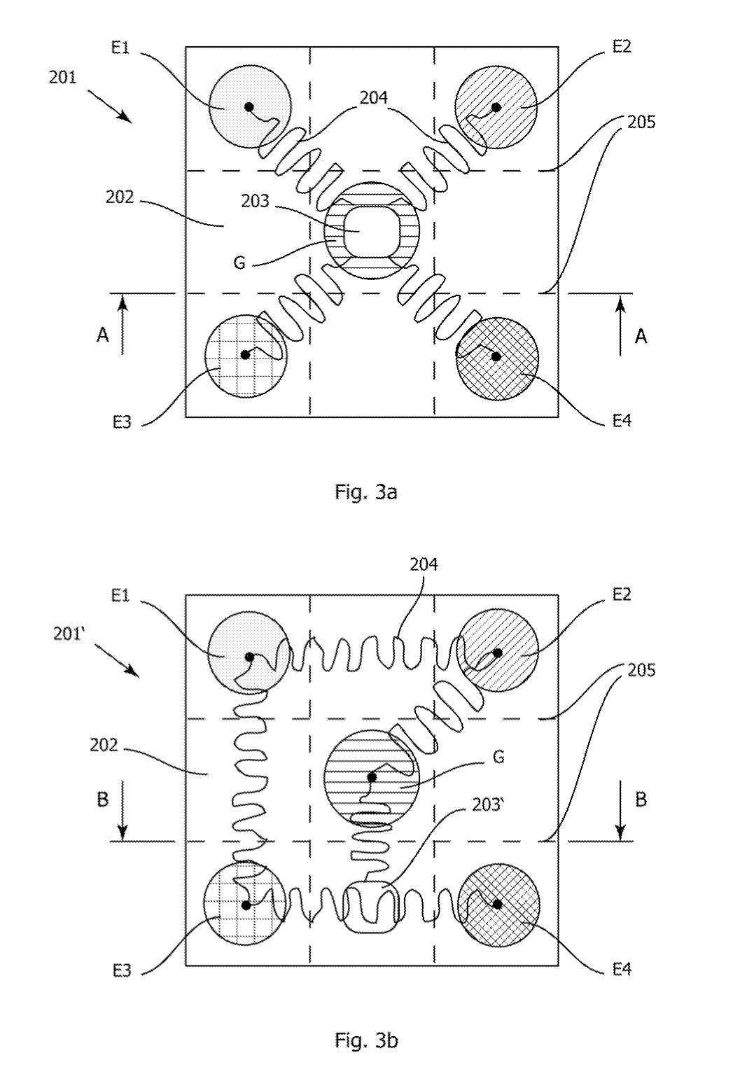

FIG. 3a shows, as an example, the top side of the support layer 202 of the first sensor device 201. The sensor device 201 comprises the lead electrodes E1 through E4 as well as the ground electrode G. Each of the electrodes E1 through E4 is each connected to the contact element 203 via a separate cable 204, which is arranged above the electrode G and is directly electrically connected to this. The cables 204 are guided in a star-shaped manner to the contact element 203 and laid on the underside of the support layer in a meander-shaped pattern or even in a loop-shaped pattern, so that after the separation of the support layer 202 at the perforation 205, a defined length is available for the cable 204. After the separation of the electrodes E1 through E4 and G, the maximum length of the cables 204 is approximately 30 cm, since a good covering of the possible applications can be achieved with this available length. However, the length of the cables 204 is not limited to this length, but rather other lengths may also be achieved.

The shown arrangement of the perforation 205 on the support layer 202 guarantees that after the separation of the support layer 202 at the perforation 205, both the electrodes E1 through E4 and the electrode G are each located on a separate section of the support layer 202. With the separation of the electrodes E1 through E4 and G, the individual cables 204, which guarantee the connection of the electrodes E1 through E4 and G to the contact element 203, are also released.

For distinguishing the electrodes E1 through E4 and G, the electrodes may be correspondingly marked. A possible type of marking is, for example, a color marking, as it also common in ECG leads. For example, E1 may be marked red, E2 black, E3 yellow, E4 green and G blue. However, other colors and/or color combinations may also be used. A descriptive marking, for example, by a numbering or by an indication of the position of these electrodes on the body of a patient is likewise possible for the electrodes E1 through E4 and G.

FIG. 3b shows, as an example, the top side of the support layer 202 of the second sensor device 201′. The sensor device 201′ likewise comprises the lead electrodes E1 through E4 as well as the ground electrode G. However, in the sensor device 201′ the contact element 203′ is correspondingly separated from the electrode G. Starting from the contact element 203′, all electrodes E1 through E4 and G are connected to the cable 204 in the sensor device 201′. The cable 204 runs from the contact element 203′ over the electrodes G, E3, E1 and E2 up to the electrode E4. For this, the cable 204 is designed as a multiwire cable with a plurality of individual shielded conductors and each of the electrodes E1 through E4 and G is each connected to one of the conductors. The cable 204 is again laid in a meander-shaped or loop-shaped pattern, for example, between the individual electrodes E1 through E4 and G.

In the sensor device 201′, with the separation of the support layer 202 at the perforation 205, not only are the electrodes E1 through E4 and G separated, the contact element 203′ is also located on a separate section of the support layer 202 after the separation. Since the support layer 202 under the contact element 203′ is designed as an adhesive pad 206, the contact element 203′ may thus also be adhered to the skin of a patient.

With regard to the arrangement and laying of the cables 204 shown in FIGS. 3a and 3b, it should be pointed out here that the arrangement and manner of laying of the cables 204 of the first sensor device 201 can also be extrapolated to the second sensor device 201′ and vice versa.

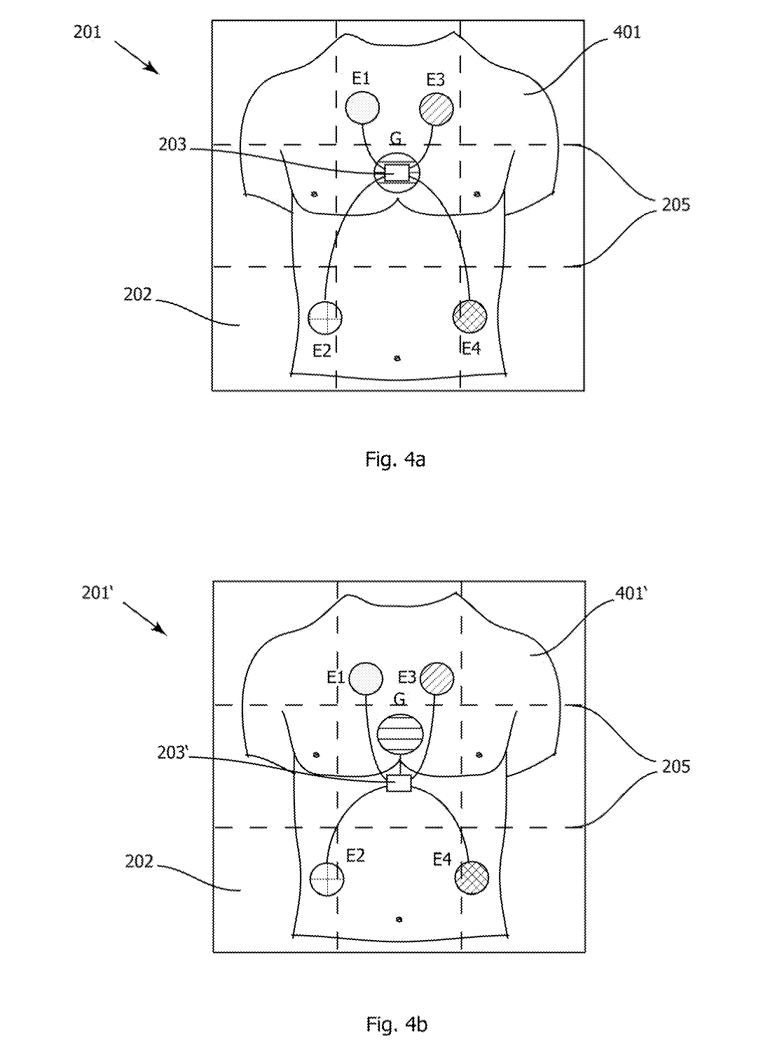

FIG. 4a schematically shows the example of a pictogram 401 for the first sensor device 201, in which the contact element 203 is arranged on the electrode G. With the pictogram 401, it is shown how the electrodes E1 through E4 and G are to be positioned on the body of a patient. It is evident from the pictogram 401 that the two electrodes E2 and E4 are positioned for detecting an SEMG signal in the area of the lower thorax on the lower right and left costal arch, respectively. An SEMG signal, which describes the muscle activity of the diaphragm as the most important respiratory muscle with inspiratory action, can then be detected via the two electrodes E2 and E4.

On the other hand, the two electrodes E1 and E3 are positioned in the area of the upper thorax over the external right and left intercostal muscles, respectively. Thus, an SEMG signal, which describes the muscle activity of the auxiliary respiratory muscles, is detected via the two electrodes E1 and E3. This has the advantage that fatigue of the diaphragm can be recognized early. A fatigue of the diaphragm can then be recognized, for example, when the auxiliary respiratory muscles, which are not active in the normal state, are activated for breathing. However, a detection of SEMG signals of the auxiliary respiratory muscles is not absolutely necessary for the monitoring of the breathing of a patient. Only the SEMG signal of the diaphragm may also be monitored.

With the paired arrangement of the electrodes E1 and E3 as well as E2 and E4, impairments of the breathing of a patient on one side can especially be diagnosed. A paired arrangement of electrodes is, however, not absolutely necessary.

It is also sufficient to position either the electrodes E1 and E2 or the electrodes E3 and E4 each on one side of the body of a patient.

The pictogram 401 has the advantage that the positioning of the individual electrodes E1 through E4 and G on the body of a patient is thus highly simplified. This effect is further enhanced by the color marking of the electrodes E1 through E4 and G. Moreover, the pictogram 401 makes possible an easy and simply assignment of the individual electrodes E1 through E4 and G to their respective lead position. This in turn facilitates the making of a diagnosis on the basis of the SEMG.

FIG. 4 schematically shows the example of a pictogram 401′ for the second sensor device 201′, in which the contact element 203′ is separated from the electrode G.

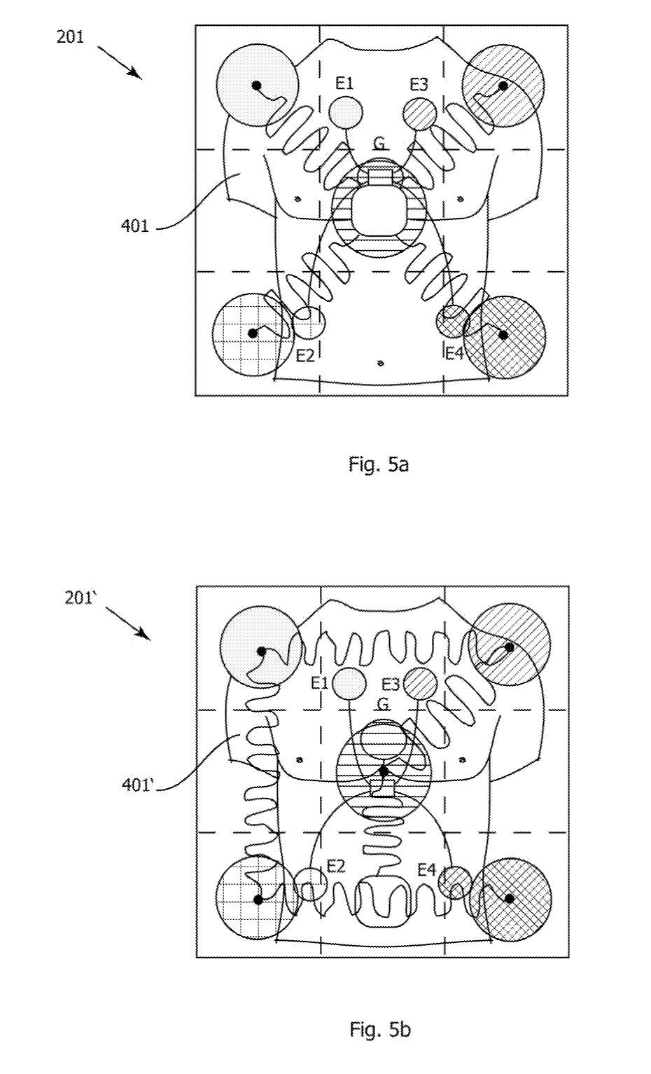

FIG. 5a schematically shows as an example the top side of the support layer 202 of the first sensor device 201 with the cabling of the electrodes E1 through E4 and G as well as with the pictogram 401. The simple assignment of the color-marked electrodes E1 through E4 and G to their respective lead position as well as the necessary positioning for the recording of an SEMG of the respiratory muscles for monitoring the breathing of a patient are shown here once again.

FIG. 5b schematically shows in a comparable manner, as an example, the top side of the support layer 202 of the second sensor device 201′ with the cabling of the electrodes E1 through E4 and G as well as with the pictogram 401′.

For a control of respiration by means of SEMG, especially in the presence of expiratory breathing efforts, it may appear meaningful to use an additional signal as a reference to the SEMG signals for the clear recognition of the two breathing phases, inspiration and expiration. This need particularly arises from the requirement to avoid a respiratory failure of a patient. The measuring of a geometric change in a human body, especially the measuring of the change in the upper body volume of a patient caused by breathing provides this reference. The measurement takes place, for example, via one or more mechanical sensors, which are connected to the body of the patient.

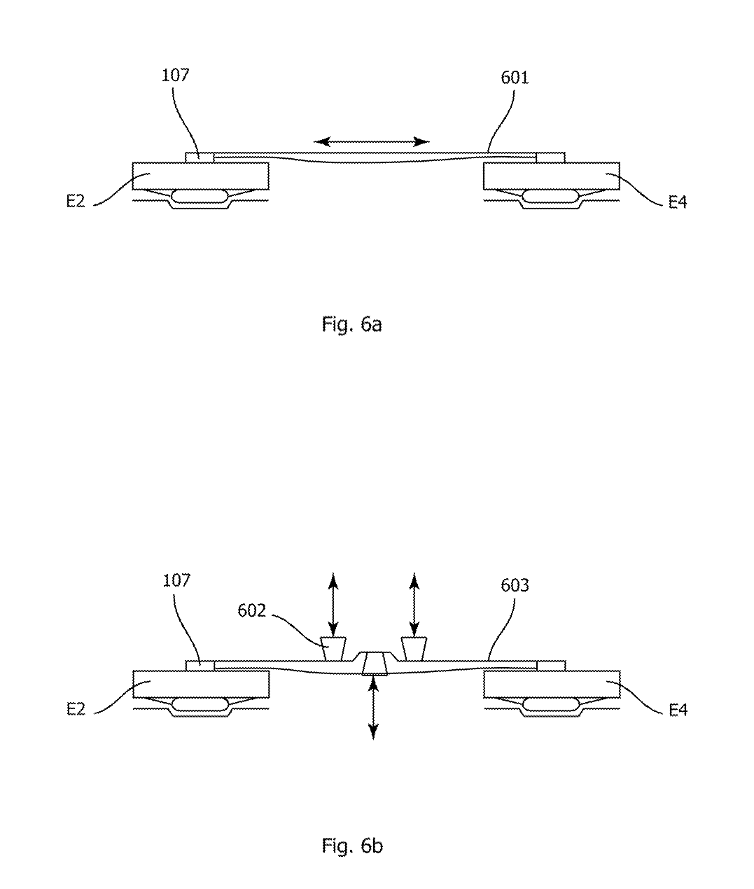

FIG. 6a schematically shows the example of a strain sensor 601 which acts as a mechanical sensor and which is arranged between the two electrodes E2 and E4. Changes in length between the two electrodes E2 and E4, which are caused by the breathing of a patient, can be determined with the strain sensor 601, which is designed, for example, as an elastic, conductive filament. Determination of the change in length is based on a measurement of resistance. The basis for this is that the change in length of the strain sensor 601 brings about a lengthening of the current path through the filament with simultaneous regeneration of the conduction cross section of the filament, so that the following formula can be applied for determining the change in length

R=ρ·L/A.

Depending on the manner of change in length of the strain sensor 601, a conclusion about the two breathing phases can be drawn. An increase in length of the strain sensor 601 denotes inspiration, while a subsequent reduction in the length of the strain sensor 601 denotes expiration.

The strain sensor 601 designed as an elastic conductive filament is not conductively suspended on the electrodes E2 and E4. For suspending, either additional, nonconductive suspensions, for example, on the contacts 107 of the electrodes E2 and E4 can be attached, or the strain sensor 601 can be connected directly to the contacts 107, provided that no electrical connection is established between the strain sensor 601 and the contacts 107.

The strain sensor 601 does not absolutely have to be arranged between the two electrodes E2 and E4. An arrangement between the two electrodes E1 and E3 is likewise possible. A use of two strain sensors 601, of which one is arranged between the electrodes E1 and E3 and the other is arranged between the electrodes E2 and E4, is also possible.

In a filament-type design of the strain sensor 601, the filament has a cross section of approximately 1 mm² and a length of approximately 15 mm. As an alternative, the strain sensor 601 may also be designed as a flat structure or comb-shaped, in order to simplify, for example, the integration of the strain sensor 601 into the sensor device 201, 201′.

FIG. 6b schematically shows the example of a mechanical sensor, which is formed from piezoelectric elements 602. The piezoelectric elements 602 are arranged above and below an elastic connecting element 603, for example, an elastic filament, which is in turn arranged between the two electrodes E2 and E4. Here, the elastic connection 603 exerts a force onto the piezoelements 602, which generate charge shifts therefrom. In a corresponding measurement of these charge shifts, a stress variation arises, which corresponds to the mechanical stress in the elastic connection 603. A conclusion about the two breathing phases can then in turn be drawn from this stress variation. An increase in the mechanical stress in the elastic connection 603 denotes inspiration, while a subsequent decrease in the mechanical stress in the elastic connection 603 denotes expiration.

Even though three piezoelectric elements 602 are shown as an example in FIG. 6b, it is also possible to use more or fewer piezoelectric elements 602 for the determination of the stress variation.

In a manner similar to the piezoelements 602, semiconductor resistors may also be used. Here, the mechanical force generated by the elastic element 603 is conducted to the semiconductor bending element and the analysis of the measurable resistance then takes place, for example, in a bridge circuit.

The above-described sensor device 201, 201′ for the myographic detection of muscle signals on the skin of a patient guarantees a simple positioning and a certain assignment of the electrodes to their respective lead position. Moreover, artefacts can be better suppressed and the two breathing phases can be better recognized in a combination of the sensor device 201, 201′ with a mechanical sensor, so that a respiratory failure of a patient is effectively prevented.

Even though the described sensor device 201, 201′ is especially suitable for myographic detection of muscle signals, such a sensor device may also be used for detecting other bioelectric signals, for example, for detecting ECG signals.

Even though the present invention was described in detail in the figures and the above description, the drawings are defined as illustrative or exemplary and not limiting; in particular, the present invention is not limited to the exemplary embodiments described. Further variants of the present invention and their design arise for the person skilled in the art from the preceding disclosure, the figures and the patent claims.

In the patent claims, terms used such as ‘comprise,’, ‘have,’ ‘include,’ ‘contain’ and the like do not rule out further elements or steps. Moreover, the use of an indefinite article does not rule out a plurality. A single device can execute the functions of a plurality of devices mentioned in the patent claims.

Reference numbers indicated in the patent claims (if any) are not to be considered as a limitation of the means and steps used.

While specific embodiments of the invention have been shown and described in detail to illustrate the application of the principles of the invention, it will be understood that the invention may be embodied otherwise without departing from such principles.

1. A sensor device for the electromyographic detection of muscle signals on the skin of a live body, the sensor device comprising:
a plurality of electrodes including at least two lead electrodes and a ground electrode, the electrodes comprising a common support layer comprising at least one perforation at which the support layer can be separated, such that after the separation, each of the electrodes is located separately on a separated section of the support layer;
a contact element; and
a shielded cable with a cable end connected to one of the electrodes and another cable end connected to the contact element, which can be connected by means of a connecting element to an analysis unit, such that signals can be transferred to the analysis unit.

2. A sensor device in accordance with claim 1, further comprising a mechanical sensor that picks up at least one geometric change in the live body.

3. A sensor device in accordance with claim 2, wherein the mechanical sensor is arranged between the two lead electrodes.

4. A sensor device in accordance with claim 2, wherein the mechanical sensor is one of a strain sensor and a piezoelectric element.

5. A sensor device in accordance with claim 1, wherein each of the electrodes is individually connected to the contact element by means of the shielded cable.

6. A sensor device in accordance with claim 1, wherein the cable comprises a multiwire cable guided from one electrode to the next electrode, whereby the cable is connected to each electrode.

7. A sensor device in accordance with claim 1, wherein the cable is laid such that after separating the support layer at the perforation, an additional length of the cable is released.

8. A sensor device in accordance with claim 7, wherein the cable is laid in a meander-shaped pattern.

9. A sensor device in accordance with claim 1, further comprising a pictogram arranged on a top side of the support layer.

10. A sensor device in accordance with claim 9, wherein the pictogram images a positioning of the electrodes on a live body and/or an assignment of the electrodes to a lead position relative to a pictured body.

11. A sensor device in accordance with claim 1, wherein the contact element is arranged above one of the electrodes.

12. A sensor device in accordance with claim 1, wherein the electrodes are color coded.

13. A method for the electromyographic recording of muscle signals on the skin of a live body, the method comprising the steps of:
providing a plurality of electrodes including at least two lead electrodes as well as one ground electrode;
providing all the electrodes arranged together on a support layer wherein the support layer is separated at least one perforation, such that after the separation, each electrode is located separately on a separated section of the support layer; and
connecting one end of the shielded cable to one of the electrodes and connecting another end of the shielded cable to a contact element and the contact element is connected by means of a connecting element to an analysis unit, such that signals can be transferred to the analysis unit.

14. A method in accordance with claim 13, wherein after separating the support layer at the perforation, an additional length of the cable is released.

15. A system for the electromyographic recording of muscle signals on the skin of a live body, the system comprising:
a sensor device comprising a plurality of electrodes including at least two lead electrodes and a ground electrode, the electrodes comprising a common support layer comprising at least one perforation, at which the support layer can be separated, such that after the separation, each of the electrodes is located separately on a separated section of the support layer, a contact element and a shielded cable with a cable end connected to one of the electrodes and another cable end connected to the contact element; and
an analysis unit, to which the sensor device is connected by means of the connecting element, such that signals can be transferred to the analysis unit.

16. A system in accordance with claim 15, wherein the sensor device further comprises a mechanical sensor that picks up at least one geometric change in the live body, wherein the mechanical sensor is arranged between the two lead electrodes.

17. A system in accordance with claim 15, wherein the cable comprises a multiwire cable guided from one electrode to the next electrode, whereby the cable is connected to each electrode.

18. A system in accordance with claim 15, wherein the cable is connected to the support layer such that after separating the support layer at the perforation, an additional length of the cable is provided.

19. A system in accordance with claim 15, wherein the sensor device further comprises a pictogram arranged on a top side of the support layer.

20. A system in accordance with claim 15, wherein the electrodes are color coded.
